(19) United States
(12) Patent Application Publication
(10) Pub. No.: US 2025/0099668 A1
(43) Pub. Date: Mar. 27, 2025

(54) COLLECTOR FOR SURGICAL SUCTION SYSTEM

(71) Applicant: TISSUEFLOW GMBH, Essen (DE)

(72) Inventor: Marcel HAVERSATH, Meerbusch (DE)

(21) Appl. No.: 18/730,826

(22) PCT Filed: Jan. 25, 2022
(86) PCT No.: PCT/EP2022/051635

§ 371 Date: Aug. 7, 2024

(51) Int. Cl. A61M 1/00

(52) U.S. Cl. CPC A61M 1/79

(57) ABSTRACT

The invention relates to a tissue collector for a surgical suction system, the tissue collector comprising a fluid inlet, a fluid outlet and a collection chamber. The collection chamber is located between the fluid inlet and the fluid outlet. At least one front filtering element and at least one rear filtering element are arranged in the collection chamber. The filtering elements are arranged one behind the other in the direction of fluid flow and each have a plurality of fluid passage openings. The diameter of the fluid passage openings in the front filtering element is larger than the diameter of the fluid passage openings in the rear filtering element. At least one carrier material is arranged on at least one filtering element. By means of the filtering elements and/or by means of the at least one carrier material, it is possible to separate autologous tissue components which are entrained in the fluid flowing through the collection chamber.

The invention relates to a tissue collector for a surgical suction system having a fluid inlet, a fluid outlet and a collection chamber between the fluid inlet and the fluid outlet. The invention further relates to a method of separating autologous tissue components from a fluid.

Tissue collectors of the above-described type are basically known from practice in different embodiments. They are used, for example, in the form of filtering systems in surgical suction systems. Surgical suction systems are used during operations on the human body, for example in orthopedic-accident surgical operations, in plastic surgery and in operations on the spinal column. In the context of these operations, blood and loose tissue components such as bone particles, cartilage and connective tissue are drawn off by the suction systems. For this purpose, the suction systems are usually equipped with a suction device in the form of a pump. Since, in most cases, follow-up systems such as autotransfusion systems are connected downstream of the systems, as a rule, the suction systems have a filtering device that prevents clogging of the subsequent systems by tissue components. The filtering systems used or their filter materials often have the disadvantage that they are rapidly clogged by aspirated tissue components and then have to be exchanged. The filtering systems are therefore usually disposable filters disposed of as soon as they are clogged. On the one hand, this is only slightly economical and, on the other hand, the collected tissue components are disposed of unused together with the disposable filter. This is from a medical point of view of little sense. The potential of the aspirated tissue components for mesenchymal tissue regeneration is not used by the disposable filters. Therefore, tissue collectors for surgical suction systems are known from practice in which the extracted tissue components can be removed together with a filter element, so that the suctioned tissue components can be further used. However, these tissue collectors generally have the disadvantage that they absorb the suctioned tissue components in a nonselective manner and the filter materials used are always subjected to a mixture of a wide variety of tissue components. Moreover, the known tissue collectors are not flexibly adaptable to specific uses of the suctioned tissue components. In particular, the known tissue collectors are often placed on a specific filter material. This is what the invention relates to.

On the other hand, the object of the invention is to provide a tissue collector of the above-described type with which the above-described disadvantages can be effectively and functionally reliably avoided and that in particular allows the flexible later use of the separated tissue components, while operating reliably over a sufficiently long period of time and with which the potential for mesenchymal tissue regeneration of the suctioned tissue components can be optimally utilized. In addition, the object of the invention is to provide a method of separating autologous tissue components from a fluid.

In order to attain these objects, the invention teaches a tissue collector for a surgical suction system where the tissue collector has a fluid inlet, a fluid outlet and a collection chamber and a collecting chamber is between the fluid inlet and the fluid outlet and at least one upstream filter element and at least one downstream filter element are in the collecting chamber and the filter elements follow one another in the fluid-flow direction and each has an array of fluid-passage openings and the diameters of the fluid-passage openings of the upstream filter element are greater than the diameters of the fluid-passage openings of the downstream filter element and there is at least one medium element upstream of at least one of the filter elements so that autologous tissue components entrained in the fluid flowing through the collecting chamber are trapped by one of the filter elements and/or by at least the medium element.

In the context of the invention, the term surgical suction system is in particular a suction system used during operations, preferably on the human body where a fluid composed for example of fat, bone marrow, blood and other tissue components may be aspirated. This fluid preferably also contains solid tissue components such as cartilage residues and bone residues and can also contain highly viscous components such as blood clots, fats and the like. Such a surgical suction system can have a suction device in the form of a pump and, remotely, a suction fitting, for example a suction nozzle, a suction nozzle or a suction tube, with which the fluid is drawn off at the surgical site. In addition, at least one collecting container for the fluid can be provided. In the context of the invention, the tissue collector according to the invention is between the suction device and the collecting container for the fluid, all in particular connected in series for removing the autologous tissue components or the solid and/or highly viscous tissue components from the aspirated fluid. Within the scope of the invention, the aspirated fluid flows preferably from the fluid inlet through the collection chamber and flows out of the fluid outlet. In this respect, “fluid-flow direction” means in particular in the direction from the fluid inlet to the fluid outlet.

The term fluid or fluid flowing through the collection chamber means, means within the scope of the invention, in particular the fluid suctioned by the surgical suction system during an operation.

Within the scope of the invention, the terms “autologous tissue component” means, in particular, endogenous tissue components that are part of the fluid extracted by the surgical suction system or carried along in this fluid. In the context of the invention, in principle, all mesenchymal and hematopoietic tissue components or tissue components can be removed from the aspirated fluid along with for example cartilage, connective tissue, muscle, bone, bone marrow, blood and tissue water.

Within the scope of the invention, the filter element in particular means an element through which a fluid can at least partially pass, while components entrained in the fluid, for example solids or highly viscous substances are at least partially collected or retained by the filter element. Within the scope of the invention, the term medium element refers in particular to a material that can trap the suctioned tissue components when passing through the collection chamber or that can absorb the tissue components. In the context of the invention, the medium element is in particular a biological medium element or a biological filter material. “Biological medium element” or in this context, “filter material” preferably means that the medium element or filter material is in the form of a tissue web or tissue scaffold may be used in the context of tissue engineering.

In the context of the invention, the term “diameter” of a fluid-passage opening means in particular that the largest diameter or the largest inside diameter of a fluid-passage opening. If the fluid-passage openings of a filter element have different diameters, the diameter of the fluid-passage openings expediently means the average diameter of the fluid-passage openings. The fact that the fluid-passage openings of a filter element are larger than the fluid-passage openings of another filter element therefore means, in particular, in the context of the invention, the mean diameter of the fluid-passage openings of the one filter element is greater than the average diameter of the fluid-passage openings of the other filter element. It is preferred that, in the case of a filter element where the diameters of the fluid-passage openings are greater than the diameters of the fluid-passage openings of another filter element, at least 70%, preferably at least 80%, preferably at least 90%, particularly preferably at least 95% of the fluid-passage openings, very particularly preferably all fluid-passage openings have a larger diameter than the fluid-passage openings of the other filter element.

According to a particularly preferred embodiment of the tissue collector according to the invention, an extreme upstream filter element having an array of fluid-passage openings is upstream of the upstream filter element in the fluid-flow direction. It is recommended that the diameter of the fluid-passage openings of the extreme upstream filter element is greater than the diameter of the fluid-passage openings of the upstream filter element. In this way, a diameter gradient is preferably obtained by the fluid-passage openings such that the diameter of the fluid-passage openings decreases from the extreme upstream filter element to the upstream filter element and toward the downstream filter element. It is preferred that the extreme upstream filter element is immediately downstream of the fluid inlet, so that advantageously no further filter element is between the fluid inlet and the extreme upstream filter element. It is further preferred that the downstream filter element is immediately upstream of the fluid outlet and that a further filter element is not between the downstream filter element and the fluid outlet. The upstream filter element is upstream of the downstream filter element in the fluid-flow direction and is preferably downstream of the extreme upstream filter element.

It is within the scope of the invention that the filter elements in the collection chamber, in particular the extreme upstream filter element and/or the upstream filter element and/or the downstream filter element, extend transversely, in particular perpendicular or substantially perpendicular to the fluid-flow direction of the fluid flowing through the collection chamber in the collection chamber. It is further preferred that the filter elements in the collection chamber, in particular the extreme upstream filter element and/or the upstream filter element and/or the downstream filter element, extend across the entire cross-sectional area of the collection chamber or substantially over the entire open cross-sectional area of the collection chamber.

It is within the scope of the invention that the fluid inlet is a connection element, preferably as a connector fitting. The connection element or the connector fitting expediently extends in regions into the collecting chamber. It is also possible for the fluid outlet to be a connection element or connector fitting. The tissue collector according to the invention can be connected to further devices of the surgical suction system by the connecting elements or fittings, for example with a connection hose. For this purpose, the connecting element or connector fitting can expediently be a conically tapering connector with a fluid passage, for example in the form of a tube that tapers conically axially away from the collection chamber.

It is within the scope of the invention that the collection chamber is round, in particular circular or substantially circular, seen in cross section. In this context, cross section means the cross section transversely, in particular perpendicularly or substantially perpendicular to the flow direction of the fluid flowing through the collecting chamber and preferably transversely, in particular perpendicularly or substantially perpendicular to a longitudinal axis of the tissue collector extending through the fluid inlet and the fluid outlet. It has been found to be successful that the filter elements in the collection chamber, in particular the extreme upstream filter element and/or the upstream filter element and/or the downstream filter element, are in plan view round or circular filter elements and, in particular, can be fitted snug with the inner surface of the collection chamber in the collection chamber.

A recommended embodiment of the tissue collector according to the invention is characterized in that the diameter of each of the fluid-passage openings of the extreme upstream filter element is 3 mm to 25 mm, preferably 4 mm to 20 mm, preferably 5 mm to 15 mm, particularly preferably 6 mm to 12 mm. Expediently, the extreme upstream filter element is a retaining grid. For this purpose, the extreme upstream filter is a lattice of strips forming the fluid-passage openings of the extreme upstream filter element or a retaining grid. It is recommended that the fluid-passage openings of the extreme upstream filter element have a polygonal shape and, in the case of the preferred embodiment of the extreme upstream filter element, as seen in the plan view, the fluid-passage openings at the outer edge of the filter element have a circularly arcuate shape.

An embodiment of very particular importance in the context of the invention is characterized in that the diameter of each of the fluid-passage openings of the upstream filter element is 0.5 mm to 8 mm, preferably 1 mm to 6 mm, preferably 1.5 mm to 5 mm, particularly preferably 2 mm to 4 mm, for example 3 mm. It has been found to be particularly useful for the upstream filter element to be in the form of a filter plate whose fluid-passage openings are expediently round holes, preferably as circular round holes. The fluid-passage openings of the upstream filter element are recommended to be uniformly distributed uniformly or substantially uniformly on the upstream filter element, in particular the filter plate. Expediently, the fluid-passage openings of the upstream filter element each have the same diameter or substantially the same diameter. It is recommended that the area proportion of the fluid-passage openings with respect to the surface of the upstream filter element is between 10% and 40%, preferably between 12% and 35%, particularly preferably between 15% and 32% and very particularly preferably between 17% and 30%. The area of the fluid-passage openings with respect to the surface of the upstream filter element means the proportion of the total open flow cross section of the fluid-passage openings to the surface of the upstream filter element without such openings.

According to a particularly recommended embodiment of the invention, the diameter of each of the fluid-passage openings of the downstream filter element is 0.2 mm to 2.5 mm, preferably 0.4 mm to 2.0 mm, preferably 0.5 mm to 1.8 mm, Particularly preferably 0.7 mm to 1.5 mm, for example 1.3 mm, it has been found that the fluid-passage openings of the downstream filter element are round holes, in particular as circular round holes. It is recommended that the fluid-passage openings of the downstream filter element each have the same diameter or substantially the same diameter.

According to one embodiment, the downstream filter element is a filter plate. A particularly preferred embodiment of the invention is characterized in that the downstream filter element is a sieve cup. Here, the sieve cup in particular means a shell-shaped filter element. The sieve cup is preferably a sieve cup a flattened hemispherical shell, in which the flattened end part expediently forms the floor of the sieve cup. The fluid-passage openings of the downstream filter element are preferably on the floor, in particular only on the floor of the sieve cup. The floor of the sieve cup is expediently a filter plate that has the fluid-passage openings and, starting from the base or filter plate, preferably extends to the inner face of the side wall. No further fluid-passage openings are preferably in the rest of the side wall. According to one embodiment, fluid-passage openings are also provided in the side wall of the sieve cup. The sieve cup preferably has a round, in particular a circular cross-sectional area, and the diameter of the cross-sectional area preferably decreases from the shell rim to the cup floor, so that the sieve cup is particularly preferably a flattened hemispherical shell. The downstream filter element, which is preferably a sieve cup, is preferably in the collection chamber of the tissue collector in such a way that its floor is juxtaposed with the fluid outlet or is upstream of the fluid outlet. The fluid then expediently flows first into the side wall, which preferably has no fluid-passage openings, and then through the floor of the sieve cup where the fluid-passage openings are. The side wall thus expediently forms a receiving space or medium space upstream of the floor of the sieve cup. It is also within the scope of the invention for the side wall of the downstream filter element a sieve cup to be designed in such a way that the sieve cup can be inserted into the downstream part of the collection chamber associated with the fluid outlet and a flow space remains expediently at least in sections between the outer surface of the side wall and the inner surface of the collecting chamber.

It is preferred that the sieve cup has a plurality of drain slots in addition to the fluid-passage openings. The drain slots are preferably in the side wall of the sieve cup. In the context of the invention, the drain slots are not fluid-passage openings. It is preferred that the drain slots extend in the direction of flow of the fluid flowing through the collecting chamber and thus each extend toward the floor of the sieve cup. Expediently, the drain slots are angularly equispaced or substantially at the same angular spacing from one another on the side wall. According to one embodiment, at least four, preferably at least six, preferably at least eight, particularly preferably at least ten and very particularly preferred at least twelve drain slots are provided in the side wall of the sieve cup. In the case of provision of the fluid-passage openings in the floor of the sieve cup, the drain slots serve as a drainage possibility for fluid exiting the collecting chamber, in particular through the flow space formed preferably between the outer surface of the side wall and the inner surface of the collecting chamber.

If the downstream filter element according to the preferred embodiment is a sieve cup, it is within the scope of the invention that the upstream filter element rests directly on the downstream filter element and in particular on the upstream dnd rim of the sieve cup. The upstream filter element then expediently delimits a space between the upstream filter element and the floor of the sieve cup and expediently surrounded by the side wall.

A particularly preferred embodiment of the tissue collector according to the invention is characterized in that there is on at least one filter element, in particular on at least the upstream filter element, preferably upstream of the upstream filter element, a flat medium element, preferably a medium membrane, particularly preferably a collagen membrane. That the flat medium element is upstream of the upstream filter element, in particular, means in the context of this invention that the two-dimensional medium element is upstream of the upstream filter element in the flow direction of the fluid flowing through the collecting chamber, in particular is directly upstream of the upstream filter element and preferably bears against the upstream face of the upstream filter element. Tissue constituents entrained in particular in the fluid are bonded to or adsorbed into the flat medium element. According to one embodiment, the flat medium element can be formed as a polymeric sheet.

It has proven successful that the flat medium element is designed with respect to its area such that it does not cover the entire surface of the upstream filter element. In this way, it is preferably ensured that some fluid-passage openings of the upstream filter element are not covered by the flat medium element and the fluid flowing through the collection chamber can pass through them and flow toward the downstream filter element. The flat medium element is preferably removable from the collecting chamber and the filtered-out tissue components on the flat medium element can be used.

In this context, it has been found that the flat medium element can be accommodated in a holding seat of a filter element, in particular of the upstream filter element and the holding seat is preferably on the face of the filter element directed toward the fluid inlet, in particular of the upstream filter element, and particularly preferably is placed on the face of the filter element facing the fluid inlet, in particular of the upstream filter element. Expediently, the holding seat is a receiving frame. The flat medium element can preferably be used in the receiving frame and thus on the filter element or locked to the upstream filter element. For this purpose, the flat medium element is expediently adapted with respect to its planar extent to the size of the receiving frame.

It has proven useful for the extreme upstream filter element to be directly upstream of the upstream filter element and preferably in contact with the holding seat of the upstream filter element. Preferably, the extreme upstream filter element is held on a frame of the upstream filter element. The upstream filter element and the extreme upstream filter element expediently form a subassembly and the holding seat or the upstream filter element preferably forms the holding seat or the upstream filter element is fitted to the receiving frame of the upstream filter element. This embodiment is based on the discovery that the holding seat or a flat medium element on the receiving frame of the upstream filter element is secured on the extreme upstream filter element, so that, in the event of pressure fluctuations in the collecting chamber, it will not fall out of the holding seat or the receiving frame toward the fluid inlet or is protected against movement in the direction of flow of the fluid flowing through the collecting chamber. In the context of this embodiment, the extreme upstream filter element thus fulfills both the function of filtering out and retaining tissue components as well as securing the position of the flat medium element on the upstream filter element and in particular in the holding seat or in the receiving frame. It is possible that the extreme upstream filter element is directly upstream of the upstream filter element and preferably bears against the upstream filter element without the interposition of a holding seat and a flat medium element can be fitted between the extreme upstream filter element and the upstream filter element.

It has proven particularly useful in this context that the upstream filter element has at least one locking extension and the extreme upstream filter element has at least one complementary locking seat so that the extreme upstream filter element can preferably be plugged onto the upstream filter element. Expediently, the upstream filter element has at least two locking extensions and the extreme upstream filter element at least two complementary locking seats and the locking extensions of the upstream filter element and the locking seats of the extreme upstream filter element expediently each lie diametrically opposite one another.

It is within the scope of the invention that a bulk-material medium element, preferably of bulk granules, is provided on at least one of the filter elements, in particular on at least of the downstream filter element, preferably on the downstream filter element. The fact that the bulk-material medium element is upstream of the downstream filter element means that the bulk-material medium element is upstream of the fluid-passage openings of the downstream filter element in the flow direction of the fluid flowing through the collecting chamber, so that the fluid preferably first impinges on the bulk-material medium element and then passes through the fluid-passage openings. Tissue components can bond to the bulk-material medium element and these tissue components can be further exploited after removal of the bulk-material medium element from the collection chamber.

One embodiment of very particular importance in the context of the invention, is characterized in that the bulk-material medium element can be in a medium space preferably upstream of the fluid-passage openings of the downstream filter element in the fluid-flow direction and the medium space is preferably formed by a side wall of a sieve cup forming the downstream filter element. The bulk-material medium element is thus preferably accommodated by or in the sieve cup. This embodiment is based on the discovery that the bulk-material medium element can then be removed from the tissue collector or the collection chamber in a simple and functionally reliable manner together with the sieve cup. Expediently, a handling element, preferably a removal pin, is on the downstream filter element, in particular on the sieve cup, with which the downstream filter element, in particular the sieve cup, can be pulled out of the collecting chamber or from the tissue collector and can in particular be pulled out together with the bulk-material medium element.

The bulk-material medium element is preferably medium granules. In principle, the bulk-material medium element can be chips, small spheres, cubes, cuboids or the like. A particularly preferred embodiment of the invention is characterized in that the bulk-material medium element is based on at least one of the group: solid hydroxyapatite, calcium phosphate, calcium carbonate, calcium sulfate-selected material and/or on the basis of at least one bioglass and/or on the basis of at least one allograft material. According to one embodiment, the bulk-material medium element is formed from a bone substitute, preferably from at least one bioresorbiter-capable bone substitute. The bulk-material medium element or the bone substitute is expediently a calcium salt selected from the group of phosphoric acid, sulfuric acid, carbonic acid-selected acid and particularly preferably is a calcium phosphate, for example beta-tricalcium phosphate. In this context, “bone substitute” means in particular a substance that can be used for filling bone defects.

A bioresorbable bone replacement in this case is a substance referred to as a bone substitute that is degradable or degraded in the body, preferably in the human body, after a certain time. Within the scope of the invention, allograft material means in particular donor tissue, for example donor bone, donor tendons and the like. According to one embodiment of the invention, the bulk-material medium element is a ceramic.

An embodiment of the tissue collector according to the invention, which has proven particularly useful, is characterized in that the collecting chamber has an inlet valve through which additives can be introduced into the collecting chamber. The inlet valve is preferably between the fluid inlet and the fluid outlet in the wall of the collection chamber. The additives that can be introduced into the collection chamber via the inlet valve are for example growth factors, antibiotics, cytokines, fibrin adhesives and the like. These additives can be introduced into the collection chamber as a function of the later intended use of the separated tissue components.

It has proven successful that the collecting chamber is an oval or ovoid or substantially oval or oval hollow body. The fluid inlet and the fluid outlet are preferably on the two ends of the collecting chamber that tapers in such an embodiment. The wall thickness of the collecting chamber is expediently 0.05 to 3 mm, preferably 0.1 to 2 mm, preferably 0.2 to 1.5 mm. In the context of the invention, a fastening ring extends around the outer surface of the collecting chamber and is centered on the collecting chamber or to the longitudinal axis of the collecting chamber. In particular, the fastening ring is spaced from the outer surface of the collecting chamber and connecting webs join it to the outer surface of the collecting chamber. The tissue collector can be fastened in the sterile region of the operating room by the fastening ring.

It has proven successful that the collecting chamber has at least one upstream part and at least one downstream part and the upstream part and the downstream part are preferably detachably connected to one another, preferably detachably screwed to one another, and the upstream part and the downstream part particularly preferably each have at least one threaded rim. Expediently, the upstream and the downstream part of the collecting chamber are cups which can be screwed together. The upstream part of the collection chamber expediently has the fluid inlet and the downstream part of the collection chamber expediently has the fluid outlet. Preferably, the threaded portion of the upstream part of the collecting chamber is provided with an internal thread and the downstream part of the collecting chamber is provided with a complementary external thread. Expediently, the threaded rims are such that the collecting chamber can be separated centrally or substantially centrally in relation to its longitudinal extent into the upstream part and the downstream part. Expediently, at least one filter element, preferably at least two filter elements, preferably at least three filter elements, and very particularly preferably all the filter elements of the tissue collector are in the separate state of the two collection-chamber parts in the downstream part of the collection chamber. Expediently, in the separated state of the two collection-chamber parts, at least the extreme upstream filter element and/or the upstream filter element and/or the extreme upstream filter element are received in the downstream part of the collection chamber.

It is within the scope of the invention that the filter elements and/or the at least one medium element of the collection chamber can be removed, so that the tissue collector is of modular construction. Expediently, the filter elements and/or the at least one medium element of the collection chamber can be removed in the separated state of the two collection-chamber parts. In the separated state of the two collection-chamber parts, the extreme upstream filter element of the collection chamber is then preferably first of all unscrewed from the downstream part of the collecting chamber preferably with the upstream filter element, optionally together with the flat medium element, of the collection chamber and the downstream part of the collecting chamber can be removed with the downstream filter element, optionally together with the bulk-material medium element, of the collection chamber or separate from the downstream part of the collection chamber. The fact that the tissue collector is of modular construction means, in particular, depending on the particular intended use of the tissue components to be separated, a specific flat medium element and/or a specific bulk-material medium element can be in the tissue collector, and in that the filter elements in the collection chamber and/or the medium element or medium elements can be removed from the tissue collector or collection chamber and can in particular be removed individually. The tissue collector can thus be adapted very flexibly to the later use of the collected tissue components, in that only individual components of the tissue collector are exchanged or removed.

In order to attain the object of the invention, the invention further teaches a method of separating autologous tissue components from a fluid, where a fluid containing autologous tissue components is drawn into a tissue collector for a surgical suction system, in particular into the above-described tissue collector, via a fluid inlet, the fluid flows through a collection chamber of the tissue collector and at least one upstream filter element and at least one downstream filter element in the collection chamber, and the filter elements are spaced one after the other in the fluid-flow direction and each have an array of fluid-passage openings and the diameters of the fluid-passage openings of the upstream filter elements are greater than the diameters of the fluid-passage openings of the downstream filter element, and upstream of the filter element there is at least one medium element and the fluid passes through the medium element and the filter elements and/or the at least one medium element are used to separate out at least part of the autologous tissue components, and the filtered fluid then flows out of the tissue collector through a fluid outlet.

Within the scope of the method according to the invention, the at least one filter element provided with autologous tissue components and/or at least one medium element provided with autologous tissue components is subsequently removed from the tissue collector. This can be done, for example, by separating or unscrewing the two collection-chamber parts from each other. After the collection chamber has been opened, a new medium element can also be introduced into the collection chamber and/or a new or purified filter element can be inserted into the collection chamber.

It is preferred that the autologous tissue components separated in the context of the method according to the invention are bone fragments and/or bone marrow and/or blood clots and/or fat. It is possible for the tissue collector according to the invention or within the scope of the method according to the invention to separate out mesenchymal and/or haematopoietic tissue components or tissue components or tissue types, in particular cartilage and/or connective tissue and/or muscle and/or bone and/or bone marrow and/or blood and/or tissue water.

It is within the scope of the invention that the fluid inlet and/or the fluid outlet and/or the collection chamber, preferably the entire tissue collector, with the exception of the at least one medium element, consists or consists essentially of at least one polymeric plastic, preferably of polymethyl methacrylate (PMMA). It is also within the scope of the invention that at least the fluid inlet and/or the fluid outlet and/or the collection chamber, preferably the entire tissue collector, with the exception of the at least one medium element, is made of at least one polymeric plastic selected from the group: “polypropylene, polyethylene, polyester, polystyrene, polymethyl methacrylate, polycarbonate.” Expediently, at least the fluid inlet and/or the fluid outlet and/or the collection chamber and preferably the entire tissue collector, with the exception of the at least one medium element, consists of at least one transparent plastic.

The invention also relates to the use of a tissue collector according to the invention in a surgical suction system for separating autologous tissue components from a suctioned fluid.

The invention is based on the discovery that with the method according to the invention or the tissue collector according to the invention can be used to filter out in a simple and functionally reliable manner autologous tissue components from fluid flowing through the tissue collector or collection chamber of the tissue collector. The tissue collector can be flexibly adapted to the later use of the separated tissue components and can in particular be provided flexibly with different medium elements. These media bind or adsorb, preferably, tissue components. Due to the interaction of the filter elements according to the invention and the at least one medium element, autologous tissue components can vary and the fluid flowing through the collection chamber can be reliably removed and the collecting chamber can be removed without any problems. It should be emphasized that the tissue collector according to the invention is suitable for the use of a wide variety of medium elements. In addition, the diameter of the fluid-passage openings of the filter elements decreases in the direction of flow of the fluid and results in a stepwise and size-specific separation of tissue components. The tissue collector according to the invention is very functionally reliable, has a simple structure and enables the problem-free exchange of filter elements and/or medium elements. It satisfies all requirements both in economic terms and in medical respects. These advantages are also achieved by little complicated measures. The tissue collector according to the invention is thus distinguished by considerable advantages in comparison with the measures known from practice.

The invention is described in more detail below with reference to a drawing illustrating only one embodiment. In the drawing:

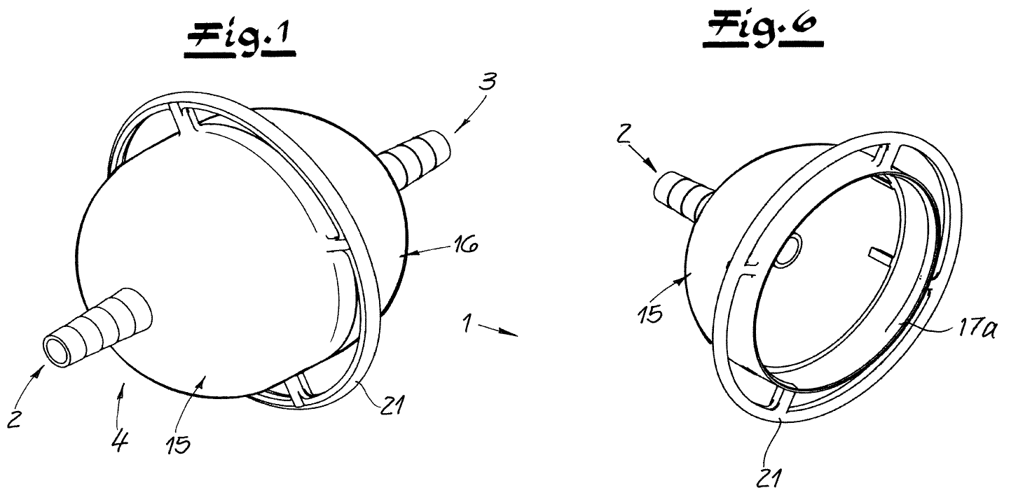

FIG. 1 is a perspective view of a tissue collector;

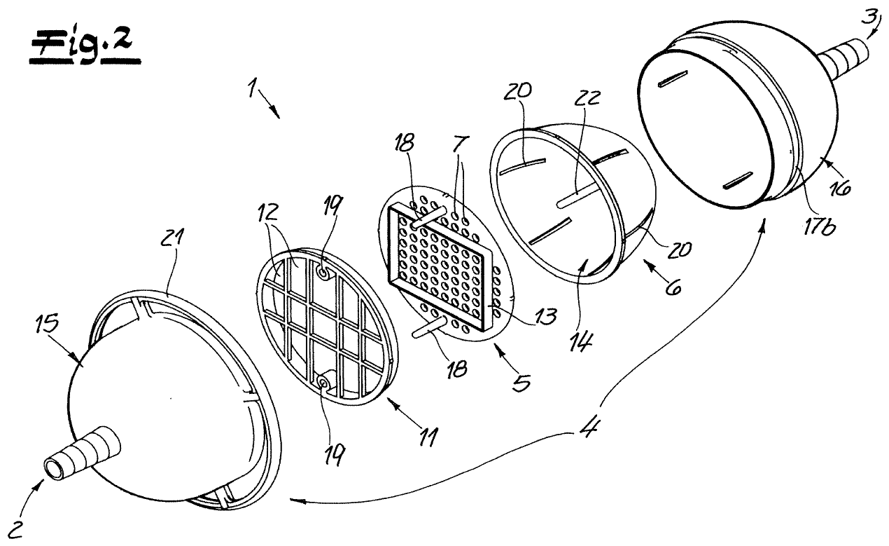

FIG. 2 is an exploded view of the collector according to FIG. 1;

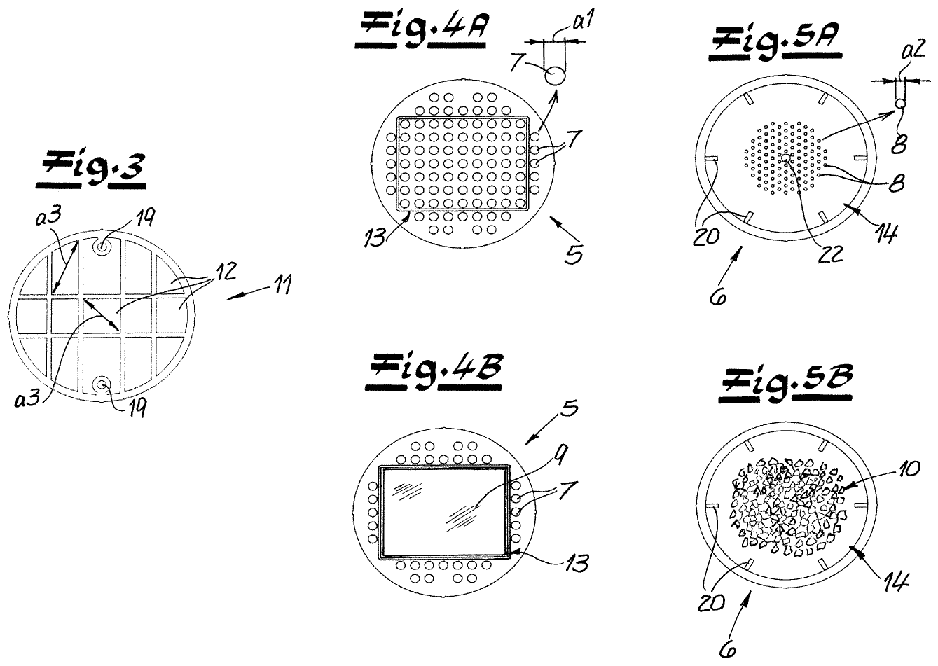

FIG. 3 shows an extreme upstream filter element according to the invention;

FIG. 4A shows an upstream filter element according to the invention;

FIG. 4B shows the filter element according to FIG. 4A with a flat medium element thereon;

FIG. 5A shows a downstream filter element or sieve cup according to the invention;

FIG. 5B shows the element according to FIG. 5A with a bulk-medium element therein; and

FIG. 6 is a perspective view of the upstream part of the collector of FIG. 2.

The drawing shows a tissue collector 1 according to the invention for a surgical suction system and having a fluid inlet 2, a fluid outlet 3 and a collection chamber 4. The collection chamber 4 is between the fluid inlet 2 and the fluid outlet 3. The fluid inlet 2 and the fluid outlet 3 are preferably and in the illustrated embodiment according to the drawing each connector fittings.

The collecting chamber 4 of the tissue collector 1 is preferably circular or substantially circular in cross section in the embodiment according to the drawing. In addition, it is of spherical shape. Further preferably and here, the collecting chamber 4 of the tissue collector 1 can be an oval or ovoid hollow body. The fluid inlet 2 on the one hand and the fluid outlet 3 on the other hand are at the two diametrally opposite ends of the collecting chamber 4. According to a preferred embodiment and here, a fastening ring 21 encircles the outer surface of the collecting chamber 4. The fastening ring 21 is recommended and here is centered on and extends coaxially to the longitudinal extent of the collecting chamber 4 or to the longitudinal axis of the collecting chamber 4 and is further preferably spaced from the outer surface of the collecting chamber 4 and is particularly preferred and here is connected to the outer surface of the collecting chamber 4 via webs. The tissue collector 1 can be fastened in the sterile region of the operating room by the fastening ring 21.

The collecting chamber 4 preferably and here has an upstream part 15 and a downstream part 16. This can be seen in particular in FIG. 2. The upstream part 15 and the downstream part 16 are expediently and detachably screwed together in the illustrated embodiment. For this purpose, the upstream part 15 of the collecting chamber 4 preferably has an internally threaded rim 17a and the downstream part 16 of the collecting chamber 4 has a complementary externally threaded rim 17b. Preferably and here according to the drawing, the upstream part 15 and the downstream part 16 of the collecting chamber 4 are formed as cups that can be screwed together and the upstream part 15 expediently has the fluid inlet 2 and the downstream part 16 has the fluid outlet 3. Further preferably, the fastening ring 21 is on the outer surface of the upstream part 15 of the collecting chamber 4.

Expediently and here according to the drawing, an extreme upstream filter element 11, an upstream filter element 5 and a downstream filter element 6 are in the collecting chamber 4. The filter elements 5, 6, and 11 succeed one another in the direction of the fluid flowing axially through the collection chamber 4 and have respective arrays of fluid-passage openings 7, 8, 12. The extreme upstream filter element 11 and the upstream filter element 5 preferably and here extend transversely, in particular perpendicular or essentially perpendicular to the flow direction of the fluid through the collection chamber 4. The extreme upstream filter element 11 and the upstream filter element 5 are expediently and here according to the drawing, flat and circular in plan view. The downstream filter element 6 preferably and here is a sieve cup and expediently and here is of circular cross-section. The filter elements 5, 6, 11 can fit snugly or substantially snugly with the inner surface of the collection chamber 4. In the embodiment according to the drawing, the filter elements 5, 6, 11 are each preferably fitted into the downstream part 16 of the collecting chamber 4 and then the upstream part 15 of the collecting chamber 4 can be placed on the downstream part 16 of the collecting chamber 4 and screwed on.

Recommended and here according to the drawing, the diameters a1 of the fluid-passage openings 7 of the upstream filter element 5 are greater than the diameters a2 of the fluid-passage openings 8 of the downstream filter element. Further preferred and here, the diameters a3 of the fluid-passage openings 12 of the furthest upstream filter element 11 are larger than the diameters a1 of the fluid-passage openings 7 of the upstream filter element 5. This can be seen in particular in FIG. 2 and FIGS. 3 to 5. In this way, a diameter gradient of the fluid-passage openings is preferably obtained, so that the diameter of the fluid-passage openings decreases from the extreme upstream filter element 11 to the downstream filter element 6. In the context of the invention, the term diameter of a fluid-passage opening means in particular the largest diameter or the largest inner diameter of a fluid-passage opening. That the diameters of the fluid-passage openings of a filter element are greater than the diameters of the fluid-passage openings of the extreme upstream filter element means in particular that the average diameter of the fluid-passage openings of the one filter element is greater than the average diameter of the fluid-passage openings of the other filter elements.

Expediently and here, the extreme upstream filter element 11 is adjacent the fluid inlet 2 and no further filter element is between the fluid inlet 2 and the extreme upstream filter element 11. Further preferably and here, the downstream filter element 6 is adjacent the fluid outlet 3 and the space between the downstream filter element 6 and the fluid outlet 3 holds no further filter element. Preferably and here, the upstream filter element 5 is in the fluid-flow direction upstream of the downstream filter element 6 and downstream of the extreme upstream filter element 11.

The diameters a3 of the fluid-passage openings 12 of each of the extreme upstream filter element 11 are preferably and here according to the drawing, 4 to 20 mm. The extreme upstream filter element 11 is preferably in the illustrated embodiment and here a retaining grid and has for this purpose very particularly preferably and here, intersecting lattice strips that form the fluid-passage openings 12 of the extreme upstream filter element 11 or of the retainer grating. The fluid-passage openings 12 of the extreme upstream filter element 11 are substantially polygonal fluid-passage openings 12 and the fluid-passage openings 12 around the outer rim of the extreme upstream filter element 11 each have a circularly arcuate outer edge.

According to a preferred embodiment of the invention, the diameters a1 of the fluid-passage openings 7 of each of the upstream filter element 5 are 1.5 mm to 5 mm, preferably 2 mm to 4 mm. In the embodiment according to the drawing, the diameters a1 of each of the fluid-passage openings 7 of the upstream filter element 5 may be approximately 3 mm. In the embodiment according to the drawing and in accordance with a preferred embodiment, the upstream filter element 5 is a filter plate. The fluid-passage openings 7 of the upstream filter element 5 are preferably and here circular round holes and each have the same diameter a1 or substantially the same diameter a1. The fluid-passage openings 7 of the upstream filter element 5 are expediently and here distributed uniformly or substantially uniformly on the upstream filter element 5 or on the filter plate.

In the context of the invention, the fluid-passage openings 8 of the downstream filter element 6 each have a diameter a2 of 0.4 mm to 2.0 mm, preferably 0.5 mm to 1.8 mm. In the embodiment according to the drawing, the diameters a2 of each of the fluid-passage openings 8 of the downstream filter element 6 may be approximately 1.5 mm. In this case, the fluid-passage openings 8 of the downstream filter element 6 expediently each have the same or substantially the same diameter a2.

The downstream filter element 6 is preferably a sieve cup in the embodiment according to the drawing. The fluid-passage openings 8 of the downstream filter element 6 are preferably only in the illustrated embodiment in the floor of the sieve cup. The rest of the sieve cup extends from the floor of the downstream filter element 6. Preferably and here according to the drawing the fluid-passage openings 8 of the downstream filter element are only on the filter plate of the floor. Thus, and here (see in particular FIG. 5A) no fluid-passage openings 8 are in the remaining side wall of the downstream filter element 6 formed as a sieve cup. The downstream filter element 6 or the sieve cup is expediently in the assembled state of the tissue collector 1 and here is in the collecting chamber 4 of the tissue collector 1 in such a way that the floor of the downstream filter element 6 is juxtaposed with the fluid outlet 3 or is upstream of the fluid outlet 3. The fluid flowing through the collecting chamber 4 then expediently flows initially through the side wall and then through the floor of the sieve cup in which the fluid-passage openings 8 are provided. When the downstream filter element 6 or the sieve cup is inserted into the downstream part 16 of the collecting chamber 4, a flow space is formed the outer surface of the sieve cup and the inner surface of the collecting chamber 4, at least in sections.

In this context, it has been found that the downstream filter element 6 or the sieve cup, in addition to the fluid-passage openings 8, has a plurality of drain slots 20 that preferably and here are only in the side wall of the downstream filter element 6 or the sieve cup. In the context of the invention, the drain slots 20 are not fluid-passage openings 8 of the downstream filter element 6. In the embodiment according to the drawing, the drain slots 20 each extend in the flow direction of the fluid flowing through the collection chamber 4 toward the floor of the sieve cup. The fluid flowing through the collection chamber 4 can run through the drain slots 20 when the fluid-passage openings 8 of the downstream filter element 6 are added.

A flat medium element 9 is preferably upstream of the upstream filter element 5 and, in the embodiment according to the drawing, the flat medium element 9 is particularly preferably a collagen membrane. The flat medium element 9 or the collagen membrane is upstream of the upstream filter element 5 in the direction of flow of the fluid flowing through the collecting chamber 4 and is preferably and here according to the drawing lies on the upstream filter element 5. Tissue constituents entrained in particular in the fluid are bound or adsorbed on the flat medium element 9. In this case, the flat medium element 9 expediently and here does not cover the entire surface of the upstream filter element 5, so that some fluid-passage openings 7 of the upstream filter element 5 remain uncovered. Preferably and here according to the drawing, the flat medium element 9 is in a holding seat 13 of the upstream filter element 5. The holding seat 13 is preferably in the embodiment is a frame expediently and here placed on the upstream face of the upstream filter element 5 directed toward the fluid inlet 2 and is in particular integrally formed on the upstream filter element 5. The flat medium element 9 can be inserted into the holding seat 13 or the receiving frame and thus locked to the upstream filter element 5. For this purpose, the flat medium element 9 is preferably adapted in terms of its area to the size of the holding seat 13 or the receiving frame in this embodiment.

A very particularly preferable embodiment of the invention is characterized in that the extreme upstream filter element 11 bears against the holding seat 13 or the receiving frame of the upstream filter element 5. Expediently and here, the upstream filter element 5 and the extreme upstream filter element 11 form a subassembly and the holding seat 13 or the receiving frame is interposed between the extreme upstream filter element 11 and the upstream filter element 5. As a result of the direct contact between the extreme upstream filter element 11 and the holding seat 13, a flat medium element 9 in the holding seat 13 or the receiving frame can be secured against falling out of the extreme upstream filter element 11. In order to form the subassembly of the extreme upstream filter element 11 and the upstream filter element 5 or for mounting, the upstream filter element 5 preferably and here has two locking extensions 18 and the extreme upstream filter element 11 has two complementary locking seats 19. In this way, the extreme upstream filter element 11 can preferably be plugged onto the upstream filter element 5.

According to a very preferable embodiment and here according to the drawing, a bulk-material medium element 10 is provided upstream of the downstream filter element 6 that is expediently and here a sieve cup. Tissue components entrained in the fluid can be bound to the bulk-material medium element 10 and these tissue components can be further used after removal of the bulk-material medium element 10 from the collection chamber 4. Expediently and here according to the drawing, the bulk-material medium element 10 can be accommodated in a medium space 14 upstream of the fluid-passage openings 8 of the downstream filter element 6 in the fluid-flow direction, and the medium space 14 expediently and here is outwardly delimited by the side wall of the cup-shaped downstream filter element 6. The bulk-material medium element is therefore preferably accommodated in the sieve cup in the embodiment. In this way, the bulk-material medium element 10 can be introduced into the sieve cup or the downstream filter element 6 in a simple and functionally reliable manner and also together with the downstream filter element 6 with which the sieve cup can be removed from the collection chamber 4. For this purpose, the downstream filter element 6 preferably has a handling element in the form of a removal pin 22 with which the downstream filter element 6 can optionally be pulled out of the collection chamber 4 together with the bulk-material medium element 10. It has already been stated that here according to the drawing, that the bulk-material medium element 10 is granules. Here the filter granules may consist or consist essentially of calcium phosphate, in particular of tricalcium phosphate or beta-tricalcium phosphate.

In the embodiment according to the drawing and preferably, the three filter elements 5, 6, 11 are in the downstream part 16 of the collecting chamber 4 when the parts 15 and 16 of the collecting chamber 4 are separated. According to a preferable embodiment, the downstream filter element 6 is first inserted into the downstream part 16 of the collecting chamber 4 and the bulk-material filter material 10 is then optionally loaded into the downstream part 16 of the collecting chamber 4 or the filter element 6 or sieve cup to fill it. The upstream filter element 5 is then preferably placed on the downstream filter element 6. If appropriate, a flat medium element 9 is then made on the upstream filter element 5, preferably in the holding seat 13 or the extreme upstream filter element 11 is then placed on the upstream filter element 5, and further preferably, the extreme upstream filter element 11 is then placed on the upstream filter element 5. The three filter elements 5, 6, 11 are then placed in the downstream part 16 of the collection chamber 4. Finally, the upstream part 15 of the collecting chamber 4 is screwed onto the downstream part 16. The filter elements 5, 6, 11 and the medium elements 9 and 10 can be removed from the collecting chamber 4, so that the tissue collector 1 overall is of modular construction. Depending on the particular intended use of the tissue components to be separated, a specific flat medium element 9 and/or a specific bulk-material medium element 10 can be in the tissue collector 1 and the filter elements 5, 6, 11 in the collecting chamber 4 and/or the medium elements 9 and 10 can be removed from the collecting chamber 4 individually. The tissue collector 1 is therefore flexibly adaptable to the later use of the tissue components.

1. A tissue collector for a surgical suction system, the tissue collector comprising
a fluid inlet,
a fluid outlet,
a collection chamber between the fluid inlet and the fluid outlet,
at least one upstream filter element in the collection chamber,
at least one downstream filter element in the collection chamber, the filter elements being one after the other in a fluid-flow direction and each having a respective array of fluid-passage openings, diameters of the fluid-passage openings of the upstream filter element being greater than diameters of fluid-passage openings of the downstream filter element, and
at least one medium element on at least one of the filter elements so the fluid flows through the filter elements and the filter elements and/or the at least one medium element filters out autologous tissue components and the filtered fluid can then flow through the outlet out of the collection chamber.

2. The tissue collector according to claim 1, wherein upstream of the upstream filter element in the fluid-flow direction there is an extreme upstream filter element having an array of fluid-passage openings having diameters that are preferably greater than diameters of the fluid-passage openings of the upstream filter element.

3. The tissue collector according to claim 2, wherein the diameters of the fluid-passage openings of the extreme upstream filter element are 3 mm to 25 mm, and the extreme upstream filter element is preferably a retaining grid.

4. The tissue collector according to claim 1, wherein the diameters of the fluid-passage openings of the upstream filter element are 0.5 mm to 8 mm and the upstream filter element is preferably a filter plate.

5. The tissue collector according to claim 1, wherein the diameters of the fluid-passage openings of the downstream filter element are 0.2 mm to 2.5 mm.

6. The tissue collector according to claim 1, wherein the downstream filter element a sieve cup having a floor provided with the fluid-passage openings of the downstream filter element.

7. The tissue collector according to claim 1, wherein the medium element is flat and upstream of the upstream filter element.

8. The tissue collector according to claim 7, wherein the flat medium element fits in a holding seat of the upstream filter element on an upstream face thereof directed toward the fluid inlet and facing the fluid inlet.

9. The tissue collector according to claim 2, wherein the extreme upstream filter element is directly upstream of the upstream filter element and on a holding seat of the upstream filter element and the upstream filter element has at least one locking extension and the extreme upstream filter element has at least one complementary locking seat so that the extreme upstream filter element is pluggable onto the upstream filter element.

10. The tissue collector according to claim 1, wherein the medium element is a bulk-material medium element on the downstream filter element.

11. The tissue collector according to claim 10, wherein the bulk-material medium element is in a medium space upstream of the fluid-passage openings of the downstream filter element in the fluid-flow direction and formed by a side wall of a sieve cup forming the downstream filter element.

12. The tissue collector according to any one of claim 10, wherein the bulk-material medium element is based on hydroxylapatite, calcium phosphate, calcium carbonate, calcium sulfate, bioglass, and/or an allograft material.

13. The tissue collector according to claim 1, wherein the collection chamber has at least one upstream part and at least one downstream part and the upstream part and the downstream part are releasably connected to each other.

14. The tissue collector according to wherein the filter elements and/or the medium element of the can be removed from collection chamber so that the tissue collector is of modular construction.

15. A method of separating autologous tissue components from a fluid, wherein a tissue collector for a surgical suction system, in particular into a tissue collector according to claim 1, draws in through a fluid inlet a fluid containing autologous tissue components and the fluid then flows through a collection chamber of the tissue collector and at least one upstream filter element and at least one downstream filter element are in the collecting chamber, the filter element and the filter elements being one after the other in the fluid-flow direction and each have an array of fluid-passage openings, wherein
the diameter of the fluid-passage openings of the upstream filter element is greater than the diameter of the fluid-passage openings of the downstream filter element and at least one medium element is upstream of at least one filter element, in particular upstream of the filter element and the fluid comprises the filter elements and at least a portion of the autologous tissue components is separated by the filter elements and/or by the at least one medium element, and the filtered fluid subsequently flows via a fluid outlet from the tissue collector.

16. The method of claim 15, wherein the autologous tissue components are bone fragments and/or bone marrow and/or blood clots and/or fat.
